(19) United States
(12) Patent Application Publication
(10) Pub. No.: US 2013/0281709 A1
(43) Pub. Date: Oct. 24, 2013

(54) PROCESS FOR PRODUCING ALPHA-HYDROXYKETONE COMPOUND

(71) Applicant: Koji Hagiya, Osaka-shi (JP)

(72) Inventor: Koji Hagiya, Osaka-shi (JP)

(73) Assignee: SUMITOMO CHEMICAL COMPANY, LIMITED, Chuo-ku, Tokyo (JP)

(21) Appl. No.: 13/979,441

(22) PCT Filed: Jan. 27, 2012
(86) PCT No.: PCT/JP2012/052437

§ 371 Date: Jul. 12, 2013

(30) Foreign Application Priority Data
Jan. 31, 2011 (JP) 2011-017658
Sep. 6, 2011 (JP) 2011-193574

(51) Int. Cl. C07C 319/20; C07D 233/20

(52) U.S. Cl. CPC C07C 319/20; C07D 233/20
USPC 548/354.1; 568/43

(57) ABSTRACT

An object of the present invention is to produce an α-hydroxyketone compound easily and effectively. Provided is a process for producing an α-hydroxyketone compound comprising a stirring step of stirring one or more aldehyde compounds or polymers thereof in the presence of a base and an imidazolinium salt represented by the formula (1):

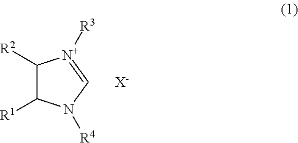

wherein R¹ and R² each independently represent a hydrogen atom, an alkyl group optionally having a substituent or an aryl group optionally having a substituent, or R¹ and R² are bound to each other to form a ring together with carbon atoms to which they bind, R³ and R⁴ each independently represent an aryl group having one or more electron withdrawing groups, and X⁻ represents an anion.

### TECHNICAL FIELD

The present application is filed, claiming the priorities based on the Japanese Patent Application Nos. 2011-017658 (filed on Jan. 31, 2011) and 2011-193574 (filed on Sep. 6, 2011), and a whole of the contents of these applications is incorporated herein by reference.

The present invention relates to a process for producing an α-hydroxyketone compound.

### BACKGROUND ART

As a process for producing an α-hydroxyketone compound by a coupling reaction of an aldehyde compound, WO 2008/19927 describes, in Table 3, a process using 1,3-bis(2,4,6-trimethylphenyl)imidazolinium-2-carboxylate as a catalyst, and a process using a catalyst prepared from 1,3-bis(2,6-diisopropylphenyl)imidazolinium chloride and a base compound, or a catalyst prepared from 1,3-bis(2,4,6-trimethylphenyl)imidazolinium chloride and a base compound.

As a process for producing the catalysts, for example, a method in which the carbonic acid gas is blown into a potassium salt of bis(trimethylsilyl)amide and a salt obtained from 1,3-bis(2,4,6-trimethylphenyl)imidazolinium chloride is described in J. of Organometallic Chemistry, 691, 5359, Scheme 3 (2006).

### DISCLOSURE OF INVENTION

### Problem to be Solved by the Invention

When a catalyst prepared from 1,3-bis(2,6-diisopropylphenyl)imidazolinium chloride and a base compound or a catalyst prepared form 1,3-bis(2,4,6-trimethylphenyl)imidazolinium chloride and a base compound is used in a process for producing an α-hydroxyketone compound, a yield of the α-hydroxyketone compound was not necessarily sufficiently satisfactory. Further, when 1,3-bis(2,4,6-trimethylphenyl)-2-carboxylate is used as a catalyst, it was necessary to use a potassium salt with bis(trimethylsilyl)amide upon preparation of the catalyst, and this was not easy from the viewpoint of handleability and safety, and was not industrially advantageous.

### Means for Solving the Problem

Under such circumstances, the present inventor intensively studied and, as a result, the following present invention is accomplished. That is, the present invention is as follows.

<1> A process for producing an α-hydroxyketone compound, comprising a stirring step of stirring one or more aldehyde compounds or polymers thereof in the presence of a base and an imidazolinium salt represented by the formula (1):

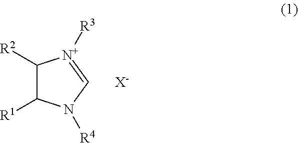

wherein
R¹ and R² each independently represent a hydrogen atom, an alkyl group optionally having a substituent or an aryl group optionally having a substituent, or R¹ and R² are bound to each other to form a ring together with carbon atoms to which they bind,
R³ and R⁴ each independently represent an aryl group having one or more electron withdrawing groups, and
X⁻ represents an anion.
<2> The process according to the above item <1>, wherein the base is at least one base selected from the group consisting of organic bases, alkali metal salts and alkaline earth metal salts.
<3> The process according to the above item <1> or <2>, wherein R³ and R⁴ each are independently an aryl group having at least one group selected from the group consisting of a halogen atom, a nitro group a cyano group, an alkoxycarbonyl group, an acyl group, and a sulfo group.
<4> The process according to the above item <1> or <2>, wherein the imidazolinium salt represented by the formula (1) is an imidazolinium salt represented by the formula (1A):

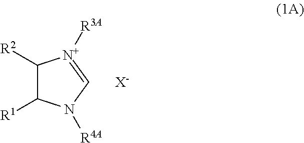

wherein
R¹, R² and X⁻ mean the same as defined above, and R^{3A} and R^{4A} each independently represent a 2,6-dichlorophenyl group optionally having a substituent, a 2,6-dibromophenyl group optionally having a substituent, a 2,6-bis(trifluoromethyl)phenyl group optionally having a substituent, a 2,6-diphenylphenyl group or a 2,6-diisopropyl-4-nitrophenyl group.
<5> The process according to any one of the above items <1> to <4>, wherein the stirring step is performed in the presence of carbon dioxide.
<6> The process according to any one of the above items <1> to <5>, wherein the aldehyde compound or polymer thereof is an aldehyde compound represented by the formula (2):

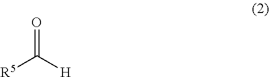

wherein
R⁵ represents a hydrogen atom or an alkyl group optionally having a substituent
or a polymer thereof.
<7> The process according to any one of the above items <1> to <5>, wherein the aldehyde compounds or polymers thereof are an aldehyde compound represented by the formula (2):

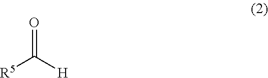

wherein
R⁵ represents a hydrogen atom or an alkyl group optionally having a substituent
or a polymer thereof and an aldehyde compound represented by the formula (4):

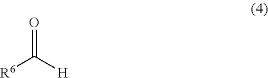

wherein
R⁶ is different from R⁵, and represents a hydrogen atom, an alkyl group optionally having a substituent, an aryl group optionally having a substituent or a heteroaryl optionally having a substituent
or a polymer thereof.
<8> The process according to the above item <7>, wherein R⁵ in the formula (2) is an alkyl group optionally having a substituent.
<9> The process according to the above item <7>, wherein the aldehyde compound represented by the formula (2) or a polymer thereof is 3-methylthiopropanal.
<10> The process according to the above item <8> or <9>, wherein the aldehyde compound represented by the formula (4) or a polymer thereof is formaldehyde or a polymer thereof, and wherein the stirring step is performed in the presence of water.
<11> An imidazolinium salt represented by the formula (1A):

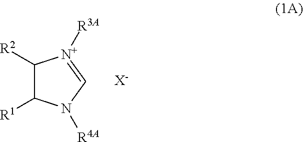

wherein
R¹, R² and X⁻ mean the same as defined above,
R^{3A} and R^{4A} each independently represent a 2,6-dichlorophenyl group optionally having a substituent, a 2,6-dibromophenyl group optionally having a substituent, a 2,6-bis(trifluoromethyl)phenyl group optionally having a substituent, a 2,6-diphenylphenyl group or a 2,6-diisopropyl-4-nitrophenyl group.
<12> The imidazolinium salt according to the above item <11>, wherein R¹ and R² are both a hydrogen atom, and R^{3A} and R^{4A} each independently represent a 2,6-dibromophenyl group, a 4-tert-butyl-2,6-dibromophenyl group, a 4-dodecyl-2,6-dibromophenyl group, a 2,4,6-tribromophenyl group, a 4-fluoro-2,6-dibromophenyl group or a 2,6-diisopropyl-4-nitrophenyl group.
<13> The imidazolinium salt according to the above item <11>, wherein R¹ and R² are both a hydrogen atom, and R^{3A} and R^{4A} each independently represent a 2,6-dibromophenyl group, a 2,4,6-tribromophenyl group, a 4-fluoro-2,6-dibromophenyl group or a 2,6-diisopropyl-4-nitrophenyl group.

### Effect of the Invention

According to the present invention, an α-hydroxyketone compound can be produced easily and effectively.

### MODE FOR CARRYING OUT THE INVENTION

Hereinafter, the present invention will be described in detail.

The present invention is characterized in that the process for producing an α-hydroxyketone compound has a stirring step of stirring one or more aldehyde compounds or polymers thereof in the presence of a base and an imidazolinium salt represented by the formula (1):

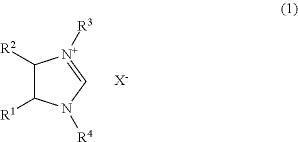

wherein all of R¹, R², R³, R⁴ and X⁻ mean the same as defined above (hereinafter, sometimes referred to as imidazolinium salt (1)).

As to R¹ and R², examples of the alkyl group include linear, branched or cyclic C₁-C₁₀ alkyl groups, such as a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group, and a menthyl group.

Examples of the substituent which the alkyl group in R¹ and R² may have include C₆-C₁₀ aryl groups optionally having a C₁-C₁₀ alkoxy group, such as a phenyl group, a naphthyl group, a 4-methylphenyl group, and a 4-methoxyphenyl group; C₁-C₁₀ alkoxy groups optionally having a fluorine atom, such as a methoxy group, an ethoxy group, a propoxy group, an isopropyloxy group, a butoxy group, an isobutyloxy group, a sec-butyloxy group, a tert-butyloxy group, and a trifluoromethyloxy group; C₁-C₁₀ alkoxy groups which have a C₆-C₁₀ aryl group optionally having a C₁-C₁₀ alkoxy group, such as a benzyloxy group, a 4-methylbenzyloxy group, and a 4-methoxybenzyloxy group; C₁-C₁₀ alkoxy groups which have a C₆-C₁₀ aryl group having a C₆-C₁₀ aryloxy group, such as a 3-phenoxybenzyloxy group; C₆-C₁₀ aryloxy groups optionally having a C₁-C₁₀ alkoxy group, such as a phenoxy group, a 2-methylphenoxy group, a 4-methylphenoxy group, and a 4-methoxyphenoxy group; C₆-C₁₀ aryloxy groups having a C₆-C₁₀ aryloxy group, such as a 3-phenoxyphenoxy group; C₂-C₁₀ acyl groups optionally having a C₁-C₁₀ alkoxy group, such as an acetyl group, a propionyl group, a benzylcarbonyl group, a 4-methylbenzylcarbonyl group, a 4-methoxybenzylcarbonyl group, a benzoyl group, a 2-methylbenzoyl group, a 4-methylbenzoyl group, and a 4-methoxybenzoyl group; a carboxy group; as well as a fluorine atom.

As to R¹ and R², examples of the alkyl group having a substituent include a fluoromethyl group, a trifluoromethyl group, a methoxymethyl group, an ethoxymethyl group, a 2-methoxyethyl group, a benzyl group, a 4-fluorobenzyl group, a 4-methylbenzyl group, a phenoxymethyl group, a 2-oxopropyl group, a 2-oxobutyl group, a phenacyl group, and a 2-carboxyethyl group.

As to R¹ and R², examples of the aryl group include C₆-C₁₀ aryl groups, such as a phenyl group, a 2-methylphenyl group, a 4-methylphenyl group, and a naphthyl group.

Examples of the substituent which the aryl group may have include C₁-C₁₀ alkyl groups having a C₁-C₁₀ alkoxy group, such as a methoxymethyl group, an ethoxymethyl group, and a 2-methoxyethyl group; C₁-C₁₀ alkoxy groups which optionally have a C₁-C₁₀ alkoxy group or a fluorine atom, such as a methoxy group, an ethoxy group, a propoxy group, an isopropyloxy group, a butoxy group, an isobutyloxy group, a sec-butyloxy group, a tert-butyloxy group, a pentyloxy group, a cyclopentyloxy group, a fluoromethyloxy group, a trifluoromethyloxy group, a methoxymethoxy group, an ethoxymethoxy group, and a 2-methoxyethoxy group; as well as halogen atoms such as a fluorine atom and a chlorine atom.

Examples of the aryl group having a substituent include a 4-chlorophenyl group and a 4-methoxyphenyl group.

In addition, R¹ and R² may be bound to each other to form a ring together with carbon atoms to which they bind. Examples of such a ring include a cyclopentane ring and a cyclohexane ring.

R³ and R⁴ each independently represent an aryl group having one or more electron withdrawing groups.

As to R³ and R⁴, examples of the aryl group include C₆-C₁₂ aryl groups, such as a phenyl group, a 2-methylphenyl group, a 4-methylphenyl group, a 2,6-diisopropylphenyl group, and a naphthyl group.

Examples of the electron withdrawing group which the aryl group have include a nitro group; a cyano group; C₂-C₁₀ alkoxycarbonyl groups such as a methoxycarbonyl group and an ethoxycarbonyl group; acyl groups such as a formyl group, an acetyl group, and a propionyl group; a sulfo group; and halogen atoms such as a fluorine atom, a chlorine atom, a bromine atom, and an iodine atom.

Examples of the aryl group having one or more electron withdrawing groups which is represented by R³ and R⁴ include C₆-C₂₀ aryl groups such as a 2-fluorophenyl group, a 2-nitronaphthyl group, a 2-cyanophenyl group, a 4-nitrophenyl group, a 2,6-dichlorophenyl group, a 2,4,6-tribromophenyl group, and a 2,6-bis(trifluoromethyl)phenyl group.

When the aryl group in R³ and R⁴ is a phenyl group, it is preferable that any one of hydrogen atoms at position 2 and position 6 on the phenyl group is substituted with an electron withdrawing and bulky group, and it is more preferable that hydrogen atoms at position 2 and position 6 on the phenyl group are both substituted with an electron withdrawing and bulky group. When hydrogen atoms at position 2 and position 6 on the phenyl group are both substituted with an electron withdrawing and bulky group, hydrogen atoms at position 2 and position 6 on the phenyl group may be substituted with the same electron withdrawing and bulky group, or may be substituted with different electron withdrawing and bulky groups.

Examples of the electron withdrawing and bulky group at position 2 and position 6 on the phenyl group include a chlorine atom, a bromine atom, an iodine atom, a nitro group, a cyano group, a carbomethoxy group, an acyl group, a sulfo group, a phenyl group, and a 3,4,5-trifluorophenyl group.

R³ is preferably a group represented by R^{3A}, and R⁴ is preferably a group represented by R^{4A}. R^{3A} and R^{4A} each independently represent a 2,6-dichlorophenyl group optionally having a substituent, a 2,6-dibromophenyl group optionally having a substituent, a 2,6-bis(trifluoromethyl)phenyl group optionally having a substituent, a 2,6-diphenylphenyl group or a 2,6-diisopropyl-4-nitrophenyl group. Examples of the substituent which the 2,6-dichlorophenyl group optionally having a substituent, the 2,6-dibromophenyl group optionally having a substituent and the 2,6-bis(trifluoromethyl)phenyl group optionally having a substituent may have at position 3, position 4 and/or position 5 on a benzene ring include the same groups as the electron withdrawing group which the aryl group represented by R³ and R⁴ has, and an alkyl group.

Examples of R^{3A} and R^{4A} preferably include a 2-trifluoromethylphenyl group, a 2,6-bis(trifluoromethyl)phenyl group, a 2,6-dichlorophenyl group, a 2,6-dibromophenyl group, a 4-tert-butyl-2,6-dibromophenyl group, a 4-dodecyl-2,6-dibromophenyl group, a 2,4,6-tribromophenyl group, a 4-fluoro-2,6-dibromophenyl group, a 2,6-diiodophenyl group, and a 2,6-diisopropyl-4-nitrophenyl group, more preferably a 2,6-dibromophenyl group, a 4-tert-butyl-2,6-dibromophenyl group, a 4-dodecyl-2,6-dibromophenyl group, a 2,4,6-tribromophenyl group and a 4-fluoro-2,6-dibromophenyl group.

Examples of the anion represented by X⁻, that is, a monovalent anion, include halide ions such as a chloride ion, a bromide ion, and an iodide ion; alkanesulfonate ions optionally having a fluorine atom as a substituent, such as a methanesulfonate ion and a trifluoromethanesulfonate ion; acetate ions optionally having a halogen atom as a substituent, such as a trifluoroacetate ion and a trichloroacetate ion; a nitrate ion; a perchlorate ion; tetrahaloborate ions such as a tetrafluoroborate ion and a tetrachloroborate ion; hexahalophosphate ions such as a hexafluorophosphate ion; hexahaloantimonate ions such as a hexafluoroantimonate ion and a hexachloroantimonate ion; pentahalostannate ions such as a pentafluorostannate ion and a pentachlorostannate ion; tetraarylborate ions optionally having a substituent, such as a tetraphenylborate ion, a tetrakis(pentafluorophenyl)borate ion, and a tetrakis[3,5-bis(trifluoromethyl)phenyl]borate ion.

Examples of such a imidazolinium salt (1) include 1,3-bis(2,6-difluorophenyl)imidazolinium chloride, 1,3-bis(2,6-dichlorophenyl)imidazolinium chloride, 1,3-bis(2,6-dibromophenyl)imidazolinium chloride, 1,3-bis(4-methyl-2,6-dibromophenyl)imidazolinium chloride, 1,3-bis(4-tert-butyl-2,6-dibromophenyl)imidazolinium chloride, 1,3-bis(4-dodecyl-2,6-dibromophenyl)imidazolinium chloride, 1,3-bis(4-fluoro-2,6-dibromophenyl)imidazolinium chloride, 1,3-bis(2,6-diiodophenyl)imidazolinium chloride, 1,3-bis(2,4,6-tribromophenyl)imidazolinium chloride, 1,3-bis[(2,6-diphenyl)phenyl]imidazolinium chloride, 1,3-bis[(2,6-trifluoromethyl)phenyl]imidazolinium chloride, and 1,3-bis[(2,6-diisopropyl)-4-nitrophenyl]imidazolinium chloride.

In addition, examples also include imidazolinium salts (1) in which the “chloride” in these imidazolinium salts (1) is substituted with “iodide”, “bromide”, “methanesulfonate”, “trifluoromethanesulfonate”, “nitrate”, “perchlorate”, “tetrafluoroborate”, “tetrachloroborate”, “hexafluorophosphate”, “hexafluoroantimonate”, “hexachloroantimonate”, “pentafluorostannate”, “pentachlorostannate”, “tetraphenylborate”, “tetrakis(pentafluorophenyl)borate”, or “tetrakis[3,5-bis(trifluoromethyl)phenyl]borate”.

Such imidazolinium salts (1) can be produced, for example, according to the method described in J. Amer. Chem. Soc., vol. 128, pp. 11768 (2006).

Examples of the base used in the present invention include at least one compound selected from the group consisting of organic bases, alkali metal salts such as alkali metal carbonate and alkaline earth metal salts such as alkaline earth metal carbonate.

Examples of such organic bases include tertiary amines such as triethylamine, trioctylamine, diisopropylethylamine, and 4-dimethylaminopyridine; nitrogen-containing cyclic compounds such as 1,8-diazabicyclo[5,4,0]-7-undecene and 1,5,7-triazabicyclo[4,4,0]-5-decene; nitrogen-containing aromatic compounds such as pyridine and imidazole; alkali metal alkoxides such as sodium methoxide and sodium ethoxide.

Examples of the alkali metal carbonate include sodium carbonate, sodium bicarbonate, potassium carbonate, potassium bicarbonate, lithium carbonate, and lithium bicarbonate.

Examples of the alkaline earth metal carbonate include magnesium carbonate and calcium carbonate.

The base used in the present invention is preferably an organic base.

The amount of the base used in the present invention is preferably from 0.1 mol to 2 mol, and more preferably from 0.5 mol to 1.5 mol per mol of the imidazolinium salt (1).

The present invention is characterized in that the process includes a stirring step of stirring one or more aldehyde compounds or polymers thereof in the presence of an imidazolinium salt (1) and a base (hereinafter, sometimes referred to as the present stirring step). A coupling reaction of the aldehyde compound or one monomer unit of a polymer thereof with the same or different aldehyde compound or one monomer unit of a polymer thereof is caused by the present stirring step (hereinafter, sometimes referred to as the present reaction), and that gives an α-hydroxyketone compound. Then, the present reaction is preferable because the selectivity in production of the α-hydroxyketone compound per unit of catalyst amount can be improved.

Hereinafter, the aldehyde compound and the present reaction will be described below, but a polymer of the aldehyde compound which is reaction-active can be similarly used in the present reaction.

The aldehyde compound used in the present invention is not limited insofar as it is a compound having at least one formyl group in its molecule. In addition, the present invention may be a homocoupling reaction in which one aldehyde compound is coupled, or a crosscoupling reaction in which different two aldehyde compounds are coupled.

Examples of the homocoupling reaction include a homocoupling reaction of an aldehyde compound represented by the formula (2):

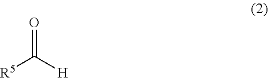

wherein R⁵ means the same as defined above (hereinafter, sometimes referred to as aldehyde (2)). By the homocoupling reaction of the aldehyde (2), an α-hydroxyketone compound represented by the formula (3):

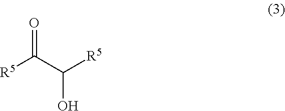

wherein R⁵ means the same as defined above (hereinafter, sometimes referred to as α-hydroxyketone (3)) is obtained.

Examples of the crosscoupling reaction include a crosscoupling reaction of the aldehyde (2) and an aldehyde compound represented by the formula (4):

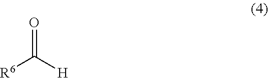

wherein R⁶ means the same as defined above (hereinafter, sometimes referred to as aldehyde (4)).

By the crosscoupling reaction of the aldehyde (2) and the aldehyde (4), an α-hydroxyketone compound represented by the formula (5):

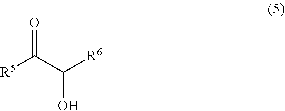

wherein R⁵ and R⁶ mean the same as defined above,
an α-hydroxyketone compound represented by the formula (6):

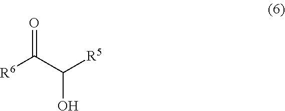

wherein R⁵ and R⁶ means the same as defined above or a mixture thereof is produced. Its production ratio is different depending on kinds of R⁵ and R⁶, and any of them is selectively produced in some cases.

As to R⁵ and R⁶, examples of the alkyl group include linear, branched or cyclic C₁-C₁₀ alkyl groups, such as a methyl group, an ethyl group, a propyl group, an isopropyl group, a butyl group, an isobutyl group, a sec-butyl group, a tert-butyl group, a pentyl group, a decyl group, a cyclopropyl group, a 2,2-dimethylcyclopropyl group, a cyclopentyl group, a cyclohexyl group, and a menthyl group.

Examples of the substituent which the alkyl group may have include C₁-C₆ alkoxy groups optionally having a fluorine atom, such as a methoxy group, an ethoxy group, a propoxy group, an isopropyloxy group, a butoxy group, an isobutyloxy group, a sec-butyloxy group, a tert-butyloxy group, and a trifluoromethyloxy group; C₁-C₁₀ alkoxy groups which have a C₆-C₁₀ aryl group optionally having a C₁-C₁₀ alkoxy group, such as a benzyloxy group, a 4-methylbenzyloxy group, and a 4-methoxybenzyloxy group; C₁-C₁₀ alkoxy groups which have a C₆-C₁₀ aryl group having a C₆-C₁₀ aryloxy group, such as a 3-phenoxybenzyloxy group; C₆-C₁₀ aryloxy groups optionally having a C₁-C₁₀ alkoxy group, such as a phenoxy group, a 2-methylphenoxy group, a 4-methylphenoxy group, and a 4-methoxyphenoxy group; C₆-C₁₀ aryloxy groups having a C₆-C₁₀ aryloxy group, such as a 3-phenoxyphenoxy group; C₂-C₁₀ acyl groups optionally having a C₁-C₁₀ alkoxy group, such as an acetyl group, a propionyl group, a benzylcarbonyl group, a 4-methylbenzylcarbonyl group, a 4-methoxybenzylcarbonyl group, a benzoyl group, a 2-methylbenzoyl group, a 4-methylbenzoyl group, and a 4-methoxybenzoyl group; C₁-C₁₀ alkylthio groups such as a methylthio group, an ethylthio group, and an isopropylthio group; C₂-C₁₀ alkoxycarbonyl groups such as a methoxycarbonyl group and an ethoxycarbonyl group; as well as halogen atoms such as a fluorine atom, a chlorine atom, and a bromine atom.

Examples of the alkyl group having a substituent include a chloromethyl group, a fluoromethyl group, a trifluoromethyl group, a methoxymethyl group, an ethoxymethyl group, a 2-methoxyethyl group, a methoxycarbonylmethyl group, a 1-ethoxycarbonyl-2,2-dimethyl-3-cyclopropyl group, and a 2-methylthioethyl group.

As to R⁶, examples of the aryl group include C₆-C₂₀ aryl groups such as a phenyl group, a 2-methylphenyl group, a 4-methylphenyl group, and a naphthyl group.

Examples of the substituent which the aryl group may have include C₁-C₁₀ alkyl groups having a fluorine atom, such as a fluoromethyl group and a trifluoromethyl group; C₁-C₁₀ alkyl groups having a C₁-C₁₀ alkoxy group, such as a methoxymethyl group, an ethoxymethyl group, and a 2-methoxyethyl group; C₁-C₁₀ alkoxy groups optionally having a fluorine atom or a C₁-C₁₀ alkoxy group, such as a methoxy group, an ethoxy group, a propoxy group, an isopropyloxy group, a butoxy group, an isobutyloxy group, a sec-butyloxy group, a tert-butyloxy group, a pentyloxy group, a cyclopentyloxy group, a fluoromethyloxy group, a trifluoromethyloxy group, a methoxymethoxy group, an ethoxymethoxy group, and a 2-methoxyethoxy group; C₆-C₁₀ aryloxy groups optionally having a C₁-C₁₀ alkoxy group, such as a phenoxy group, a 2-methylphenoxy group, a 4-methylphenoxy group, and a 4-methoxyphenoxy group; C₆-C₁₀ aryloxy groups having a C₆-C₁₀ aryloxy group, such as a 3-phenoxyphenoxy group; C₂-C₁₀ acyl groups optionally having a C₁-C₁₀ alkoxy group, such as an acetyl group, a propionyl group, a benzylcarbonyl group, a 4-methylbenzylcarbonyl group, and a 4-methoxybenzylcarbonyl group; a nitro group; halogen atoms such as a fluorine atom and a chlorine atom; as well as C₁-C₆ alkylenedioxy groups such as a methylenedioxy group.

Examples of the aryl group having a substituent include a 4-chlorophenyl group, a 4-methoxyphenyl group, and a 3-phenoxyphenyl group.

As to R⁶, examples of the heteroaryl group include C₄-C₁₀ heteroaryl groups having at least one hetero atom such as a nitrogen atom, an oxygen atom, and a sulfur atom, such as a pyridyl group, a furyl group, and a 5-methylfuryl group.

Examples of the substituent which the heteroaryl group may have include C₁-C₁₀ alkyl groups having a fluorine atom, such as a fluoromethyl group and a trifluoromethyl group; C₁-C₁₀ alkyl groups having a C₁-C₁₀ alkoxy group, such as a methoxymethyl group, an ethoxymethyl group, and a 2-methoxyethyl group; C₁-C₁₀ alkoxy groups optionally having a fluorine atom or a C₁-C₁₀ alkoxy group, such as a methoxy group, an ethoxy group, a propoxy group, an isopropyloxy group, a butoxy group, an isobutyloxy group, a sec-butyloxy group, a tert-butyloxy group, a pentyloxy group, a cyclopentyloxy group, a fluoromethoxy group, a trifluoromethoxy group, a methoxymethoxy group, an ethoxymethoxy group, and a 2-methoxyethoxy group; C₆-C₁₀ aryloxy groups optionally having a C₁-C₁₀ alkoxy group, such as a phenoxy group, a 2-methylphenoxy group, a 4-methylphenoxy group, and a 4-methoxyphenoxy group; C₆-C₁₀ aryloxy groups having a C₆-C₁₀ aryloxy group, such as a 3-phenoxyphenoxy group; C₂-C₁₀ acyl groups optionally having a C₁-C₁₀ alkoxy group, such as an acetyl group, a propionyl group, a benzylcarbonyl group, a 4-methylbenzylcarbonyl group, and a 4-methoxybenzylcarbonyl group; a nitro group; as well as halogen atoms such as a fluorine atom and a chlorine atom.

Examples of the heteroaryl group having a substituent include a 2-chloropyridyl group.

Examples of the aldehyde (2) include aliphatic aldehydes such as formaldehyde, acetaldehyde, propionaldehyde, n-butylaldehyde, cyclopentanecarbaldehyde, cyclohexanecarbaldehyde, 2-methylpropanal, 2,2-dimethylpropanal, 3-methylthiopropanal, 2,2-dimethylbutanal, 1-methylcyclohexanecarbaldehyde, 2,2-dimethylnonanal, and methyl 2,2-dimethyl-3-oxopropanoate. In addition, a polymer of formaldehyde such as paraformaldehyde can be used as the aldehyde (2). Furthermore, the aldehyde may be used in the form where it is present with water, such as formalin.

Examples of the aldehyde (4) include the above-mentioned aliphatic aldehydes; aromatic aldehydes such as benzaldehyde, 4-fluorobenzaldehyde, 4-nitrobenzaldehyde, 3-bromobenzaldehyde, 2-chlorobenzaldehyde, 4-methylbenzaldehyde, 3-methoxybenzaldehyde, 3,4,5-trimethoxybenzaldehyde, 3,4-methylenedioxybenzaldehyde, and 1-naphthoaldehyde; as well as heteroaromatic aldehydes such as picolinaldehyde and nicotinaldehyde. In addition, a polymer of formaldehyde such as paraformaldehyde can be used as the aldehyde (4). Furthermore, the aldehyde can be used in the form where it is present with water, such as formalin.

In the present invention, as the aldehyde compound, commercially available aldehyde compounds may be used, or aldehyde compounds produced by the known method may be used.

The present stirring step is preferably carried out in the presence of a solvent.

Examples of the solvent used in the present stirring step include aromatic hydrocarbon solvents such as toluene, xylene, and chlorobenzene; aliphatic hydrocarbon solvents such as pentane, hexane, and heptane; halogenated hydrocarbon solvents such as dichloromethane, dichloroethane, and chloroform; ether solvents such as diethyl ether, methyl tert-butyl ether, and tetrahydrofuran; ester solvents such as ethyl acetate; amide solvents such as N,N-dimethylformamide and N,N-dimethylacetamide; and alcohol solvents such as methanol and ethanol.

When an aqueous solution such as formalin is used, that is, formaldehyde which is present with water in advance, is used as the aldehyde compound for the present stirring step, a reaction can be effectively performed by using a solvent having no compatibility with water. As the solvent having no compatibility with water, the above-mentioned aromatic hydrocarbon solvents; aliphatic hydrocarbon solvents; and halogenated hydrocarbon solvents are preferably used.

The amount of the solvent used, in view of a volume efficiency, is practically preferably 100 parts by weight or less per part by weight of a total amount of the aldehyde compounds or polymers thereof.

The present stirring step is preferably carried out in the presence of carbon dioxide. The carbon dioxide for use in the present stirring step may be in either form of gaseous carbon dioxide, a solid carbon dioxide (i.e. dry ice) or supercritical carbon dioxide. The gaseous carbon dioxide may be diluted with an inert gas such as nitrogen.

The amount of carbon dioxide arbitrarily used in the present stirring step is preferably 1 mol or more per mol of a base. Although the upper limit of the amount is not limited, it is, for example, 1000 mol or less from the viewpoint of productivity.

The present stirring step is carried out, for example, by a method in which one or more aldehyde compounds or polymers thereof, an imidazolinium salt (1), a base and optionally a solvent are put into a reaction container equipped with a normally used stirring means, and then the mixture is stirred. An order of mixing one or more aldehyde compounds or polymers thereof, an imidazolinium salt (1), a base and an optionally used solvent is not limited, but a method in which one or more aldehyde compounds or polymers thereof, an imidazolinium salt (1) and optionally a solvent are put into the container and the mixture is stirred, and then, a base is added thereto and continuously stirred them, is preferably used. A method in which the present stirring step is carried out in a reaction container under carbon dioxide atmosphere is more preferably used.

A temperature in the reaction container for the present stirring step may be from −20° C. to 200° C.

When one kind of aldehyde compound or a polymer thereof is used in the present invention, the amount of the imidazolinium salt (1) to be used is preferably from 0.0001 mol to 0.2 mol, and more preferably from 0.001 mol to 0.1 mol per mol of the aldehyde compound or a monomer unit of a polymer thereof.

When two kinds of aldehyde compounds or polymers thereof are used in the present invention, the amount of the imidazolinium salt (1) used is preferably from 0.0001 mol to 0.2 mol, and more preferably from 0.001 mol to 0.1 mol, and the amount of one aldehyde compound or monomer unit of a polymer thereof is one mol or more, based on one mol of aldehyde compound in which the amount as a molar quantity is smaller among the aldehyde compounds used, or polymer thereof.

The present stirring step may be carried out under normal pressure or increased pressure. The present stirring step may be also carried out under increased pressure with gaseous carbon dioxide.

Progress of the present reaction which is carried out by the present stirring step can be confirmed by the analytical means such as gas chromatography, high performance liquid chromatography, thin-layer chromatography, NMR, and IR.

After completion of the present reaction performed by the present stirring step, an α-hydroxyketone compound can be brought out, for example, by concentrating the resulting reaction mixture. The obtained α-hydroxyketone compound may be further purified, for example, by the purification means such as distillation and column chromatography.

Examples of the thus obtained α-hydroxyketone compound include 2-hydroxyacetaldehyde, 3-hydroxy-2-butanone, 4-hydroxy-3-hexanone, 1,6-dimethylthio-4-hydroxy-3-hexanone, 5-hydroxy-4-octanone, 2-hydroxy-1-phenylethanone, 2-hydroxy-1-(4-chlorophenyl)ethanone, 2-hydroxy-1-(2-fluorophenyl)ethanone, 4-(methylthio)-2-oxo-1-butanol, 1-hydroxy-2-propanone, 1-hydroxy-2-butanone, 1-hydroxy-2-pentanone, and 2-hydroxy-1-cyclohexanone.

The present invention is industrially advantageous since the α-hydroxyketone compound can be produced easily and effectively.

### EXAMPLES

Hereinafter, the present invention will be described in more detail by way of Examples.

### Example 1

### Synthesis of 1,3-bis(2,6-dibromophenyl)imidazolinium chloride

A 200 mL flask replaced with nitrogen was charged with 10 g of 2,6-dibromoaniline, 120 g of chloroform and 4.8 g of triethylamine. To the resulting mixture was added dropwise 6 g of oxalyl chloride at 0° C. over 30 minutes. The resulting mixture was stirred at 0° C. for 2 hours and, thereafter, was stirred at room temperature for 18 hours. By adding 100 g of water to the resulting reaction mixture, a crystal was precipitated. Then, after the precipitated crystal was recovered by filtering operation, the obtained substance was washed with 10 g of water and 20 g of diethyl ether, and further dried to obtain 9.2 g of a white crystal. It was confirmed by gas chromatography/mass spectrometry (GC-MS) that the resulting white crystal is N,N′-bis(2,6-dibromophenyl)ethanediamide. Yield: 84%.

MS (m/z): 555 (M+)

After a 200 mL autoclave made of stainless was charged with 2.5 g of the above-obtained N,N′-bis(2,6-dibromophenyl)ethanediamide and 30 mL of BH₃.tetrahydrofuran (1M solution), the mixture was heated and stirred at 75° C. for 16 hours. After cooled to room temperature, the reaction solution was added in portions to a mixed liquid of 80 g of methanol and 5 g of 35% hydrochloric acid, and stirred. Lower-boiling substances were distilled off from the resulting reaction liquid, 100 g of methanol was further added to the residue, and lower-boiling substances were distilled off again to obtain 2.1 g of a white crystal. Yield: 82%

It was confirmed by GC-MS that the resulting crystal is N,N′-bis(2,6-dibromophenyl)-1,2-ethanediamine hydrochloride.

MS (m/z): 528 (M+, free amine)

A 200 mL flask replaced with nitrogen was charged with 2 g of the above-obtained N,N′-bis(2,6-dibromophenyl)-1,2-ethanediamine hydrochloride, and 24 g of triethyl orthoformate. After the resulting mixture was refluxed for 1 hour, the mixture was cooled to room temperature to precipitate a crystal. Then, after the resulting crystal was recovered by filtering operation, the obtained substance was washed with 5 g of tetrahydrofuran, and dried to obtain 560 mg of a white crystal. It was confirmed by ¹H-NMR that the resulting crystal is 1,3-bis[(2,6-dibromo)phenyl]imidazolinium chloride. Yield: 27%

¹H-NMR (δ/ppm, DMSO-d6, tetramethylsilane standard): 4.60 (s, 4H), 7.5 (m, 2H), 8.0 (m, 4H), 9.81 (s, 1H)

### Example 2

### Synthesis of 1,3-bis(2,4,6-tribromophenyl)imidazolinium chloride

A 300 mL flask replaced with nitrogen was charged with 25 g of 2,4,6-tribromoaniline, 200 g of chloroform and 9.2 g of triethylamine. To the resulting mixture was added dropwise 11.5 g of oxalyl chloride at 0° C. over 30 minutes. The resulting mixture was stirred at 0° C. for 2 hours and, thereafter, was further stirred at room temperature for 18 hours. By adding 100 g of water to the resulting reaction mixture, a crystal was precipitated. Then, after the precipitated crystal was recovered by filtering operation, the obtained substance was washed with 10 g of water and 20 g of diethyl ether, and further dried to obtain 20.4 g of a white crystal. It was confirmed by GC-MS that the resulting white crystal is N,N′-bis(2,4,6-tribromophenyl)ethanediamide. Yield: 76%.

MS (m/z): 713 (M+)

After a 200 mL autoclave made of stainless was charged with 10.1 g of the above-obtained N,N′-bis(2,4,6-tribromophenyl)ethanediamide and 85 mL of BH3.tetrahydrofuran (1 M solution), the mixture was heated and stirred at 75° C. for 16 hours. After cooled to room temperature, the reaction liquid was added in portions to a mixed liquid of 170 g of methanol and 8.5 g of 35% hydrochloric acid, and stirred. Lower-boiling substances were distilled off from the resulting reaction liquid, 150 g of methanol was further added to the residue, and lower-boiling substances were distilled off again to obtain 9.1 g of a white crystal. Yield: 89%

It was confirmed by GC-MS that the resulting crystal is N,N′-bis(2,4,6-tribromophenyl)-1,2-ethanediamine hydrochloride.

MS (m/z): 685 (M+, free amine)

A 200 mL flask replaced with nitrogen was charged with 9 g of the above-obtained N,N′-bis(2,4,6-tribromophenyl)-1,2-ethanediamine hydrochloride and 100 g of triethyl orthoformate. After the resulting mixture was refluxed for 1 hour, the mixture was cooled to room temperature to precipitate a crystal. Then, after the precipitated crystal was recovered by filtering operation, the obtained substance was washed with 10 g of tetrahydrofuran, and dried to obtain 3.1 g of a white crystal. It was confirmed by ¹H-NMR that the resulting crystal is 1,3-bis[(2,4,6-tribromo)phenyl]imidazolinium chloride. Yield: 32%

¹H-NMR (δ/ppm, DMSO-d6, tetramethylsilane standard): 4.66 (s, 4H), 8.3 (s, 4H), 9.70 (s, 1H)

### Example 3

### Synthesis of 1,3-bis[(2,6-diisopropyl)-4-nitrophenyl]imidazolinium chloride

To a 100 mL flask replaced with nitrogen were added 1.0 g of commercially available 1,3-bis[(2,6-diisopropyl)phenyl]imidazolinium chloride and 10 mL of concentrated sulfuric acid, and the mixture was cooled to 0° C. Then, 7.5 mL of 68% nitric acid was slowly added thereto, thereafter, a temperature was elevated to room temperature, and the mixture was stirred for 1.5 hours, and then, this reaction liquid was added to 100 mL of ice water to precipitate a crystal. Then, after the precipitated crystal was recovered by filtering operation, the obtained substance was washed with 20 g of water and 20 g of diethyl ether, and dried to obtain 1.52 g of a white crystal. It was confirmed by ¹H-NMR that the resulting crystal is 1,3-bis[(2,6-diisopropyl)-4-nitrophenyl]imidazolinium chloride.

Yield: 98%

¹H-NMR (δ/ppm, DMSO-d6, tetramethylsilane standard): 1.1-1.5 (m, 12H), 2.9-3.1 (m, 2H), 3.3-3.5 (m, 2H), 4.6-4.8 (m, 4H), 7.7 (d, 2H), 7.9 (d, 2H), 9.70 (s, 1H)

### Example 4

### Synthesis of 1,3-bis[(4-dodecyl-2,6-dibromo)phenyl]imidazolinium chloride

A 300 mL flask replaced with nitrogen was charged with 9.3 g of 4-dodecylaniline, 150 g of chloroform and 4.3 g of triethylamine. To the resulting mixture was added dropwise 5.4 g of oxalyl chloride at 0° C. over 30 minutes. The resulting mixture was stirred at 0° C. for 2 hours and, thereafter, was further stirred at room temperature for 18 hours. When 100 g of water was added to the resulting reaction mixture, a crystal was not precipitated, and an oil layer became the emulsion state and was separated from the aqueous layer. After the aqueous layer was separated, the oil layer was further washed with 50 g of water, and the layers were separated. Then, the oil layer was concentrated, and dried to obtain 11.4 g of a light brown crystal (N,N′-bis(4-dodecylphenyl)ethanediamide).

After a 200 mL of an autoclave made of stainless was charged with 9 g of the above-obtained N,N′-bis(4-dodecylphenyl)ethanediamide and 100 mL of BH3.tetrahydrofuran (1M solution), the mixture was heated and stirred at 75° C. for 16 hours. After cooled to room temperature, the reaction liquid was added in portions to a mixed liquid of 170 g of methanol and 8.5 g of 35% hydrochloric acid, and stirred. Lower-boiling substances were distilled off from the resulting reaction liquid, 150 g of methanol was further added to the residue, and lower-boiling substances were distilled off again to obtain 5.0 g of a white crystal. Yield: 58%

It was confirmed by ¹H-NMR that the resulting crystal is N,N′-bis(4-dodecylphenyl)-1,2-ethanediamine hydrochloride.

¹H-NMR (δ/ppm, CDCL3, tetramethylsilane standard): 0.95 (m, 6H), 1.4-1.7 (m, 40H), 2.48-2.54 (m, 4H), 3.90 (s, 4H), 7.0-7.3 (m, 8H)

A 50 mL flask replaced with nitrogen was charged with 2 g of the above-obtained N,N′-bis(4-dodecylphenyl)-1,2-ethanediamine hydrochloride and 20 g of chloroform, and then, 2.5 g of N-bromosuccinimide was added thereto in portions. A temperature of the resulting slurry was elevated to 70° C., and the slurry was stirred at the same temperature for 1 hour. After cooled to room temperature, 20 g of water was added, the chloroform layer was washed with water, and separated with a separatory funnel, and the same operation was repeated once more. The chloroform layer was dried with magnesium sulfate, and the solvent was distilled off to obtain 3.2 g of a brownish oil (N,N′-bis(4-dodecyl-2,6-dibromophenyl)-1,2-ethanediamine hydrochloride).

A 100 mL flask replaced with nitrogen was charged with the all amount of the above-obtained N,N′-bis(4-dodecyl-2,6-dibromophenyl)-1,2-ethanediamine hydrochloride, 20 g of triethyl orthoformate, and 720 mg of concentrated hydrochloric acid. After the resulting mixture was refluxed for 1 hour while removing the evolved ethanol, the mixture was cooled to room temperature to precipitate a crystal. Then, after the precipitated crystal was recovered by filtering operation, the obtained substance was washed with 10 g of diethyl ether, and dried to obtain 1.5 g of a white crystal. It was confirmed by ¹H-NMR that the resulting crystal is 1,3-bis[(4-dodecyl-2,6-dibromo)phenyl]imidazolinium chloride.

Yield: 45% (by two steps of bromination and conversion into imidazolinium salt)

¹H-NMR (δ/ppm, CDCL3, tetramethylsilane standard): 0.90 (m, 6H), 1.2-1.8 (m, 40H), 2.50-2.56 (m, 4H), 4.70 (s, 4H), 7.49 (bs, 4H), 10.19 (s, 1H)

### Example 5

### Synthesis of 1,3-bis[(4-methyl-2,6-dibromo)phenyl]imidazolinium chloride

A 50 mL flask replaced with nitrogen was charged with 2 g of N,N′-bis(4-methylphenyl)-1,2-ethanediamine hydrochloride and 20 g of chloroform, and then, 5.7 g of N-bromosuccinimide was added thereto in portions, and the resulting slurry was stirred at room temperature for 1 hour. After the reaction, 20 g of water was added, the chloroform layer was washed with water, and separated with a separatory funnel, and the same operation was repeated once more. After the chloroform layer was dried with magnesium sulfate, the solvent was distilled off to obtain 3.5 g of a light yellow crystal. It was confirmed by GC-MS that the resulting crystal is N,N′-bis(4-methyl-2,6-dibromophenyl)-1,2-ethanediamine hydrochloride.

MS (m/z): 555 (M+, free amine)

A 100 mL flask replaced with nitrogen was charged with the all amount of the above-obtained N,N′-bis(4-methyl-2,6-dibromophenyl)-1,2-ethanediamine hydrochloride, 16 g of triethyl orthoformate, and 1.6 g of concentrated hydrochloric acid. After the resulting mixture was refluxed for 1 hour while removing the evolved ethanol, the mixture was cooled to room temperature to precipitate a crystal. Then, after the precipitated crystal was recovered by filtering operation, the obtained substance was washed with 10 g of diethyl ether, and dried to obtain 1.04 g of a white crystal. It was confirmed by ¹H-NMR that the resulting crystal is 1,3-bis[(4-methyl-2,6-dibromo)phenyl]imidazolinium chloride.

Yield: 27% (by two steps of bromination and conversion into imidazolinium salt)

¹H-NMR (δ/ppm, CDCL3, tetramethylsilane standard): 2.48 (s, 6H), 4.67 (s, 4H), 7.48 (bs, 4H), 10.01 (s, 1H)

### Example 6

### Synthesis of 1,3-bis(4-t-butyl-2,6-dibromophenyl)imidazolinium chloride

A 300 mL flask replaced with nitrogen was charged with 10 g of 4-t-butyl-2,6-dibromoaniline, 120 g of chloroform and 4.0 g of triethylamine. To the resulting mixture was added dropwise 5.0 g of oxalyl chloride at 0° C. over 30 minutes. The resulting mixture was stirred at 0° C. for 2 hours and, thereafter, was further stirred at room temperature for 18 hours. When 100 g of water was added to the resulting reaction mixture, a crystal was not precipitated, and the oil layer became the oil state and was separated from the aqueous layer. After the aqueous layer was separated, the oil layer was further washed with 50 g of water, and was separated. Then, the oil layer was concentrated, and dried to obtain 10.5 g of a light brown crystal (N,N′-bis(4-t-butyl-2,6-dibromophenyl)ethanediamide).

After a 200 mL autoclave made of stainless was charged with 5 g of the above-obtained N,N′-bis(4-t-butyl-2,6-dibromophenyl)ethanediamide and 100 mL of BH3.tetrahydrofuran (1M solution), the mixture was heated and stirred at 75° C. for 16 hours. After cooled to room temperature, the reaction liquid was added in portions to a mixed liquid of 170 g of methanol and 8.5 g of 35% hydrochloric acid, and stirred. Lower-boiling substances were distilled off from the resulting reaction liquid, 150 g of methanol was further added to the residue, and lower-boiling substances were distilled off again to obtain 5.5 g of a brownish oil (N,N′-bis(4-t-butyl-2,6-dibromophenyl)-1,2-ethanediamine hydrochloride).

A 100 mL flask replaced with nitrogen was charged with the all amount of the above-obtained N,N′-bis(4-t-butyl-2,6-dibromophenyl)-1,2-ethanediamine hydrochloride, 20 g of triethyl orthoformate, and 1.5 g of concentrated hydrochloric acid. After the resulting mixture was refluxed for 1 hour while removing the evolved ethanol, the mixture was cooled to room temperature to precipitate a crystal. Then, after the precipitated crystal was recovered by filtering operation, the obtained substance was washed with 10 g of diethyl ether, and dried to obtain 900 mg of a white crystal. It was confirmed by ¹H-NMR that the resulting crystal is 1,3-bis[(4-t-butyl-2,6-dibromo)phenyl]imidazolinium chloride. Yield: 18% (by two steps of reduction and conversion into imidazolinium salt)

¹H-NMR (δ/ppm, CDCL3, tetramethylsilane standard): 1.32 (s, 18H), 4.60 (bs, 4H), 7.61 (s, 4H), 10.14 (s, 1H)

### Example 7

### Synthesis of 1,3-bis(2,6-dibromo-4-fluorophenyl)imidazolinium chloride

A 200 mL flask replaced with nitrogen was charged with 10.8 g of 2,6-dibromo-4-fluoroaniline, 108 g of tetrahydrofuran and 4.1 g of triethylamine. To the resulting mixture was added dropwise 5.1 g of oxalyl chloride at 0° C. over 30 minutes. The resulting mixture was stirred at 0° C. for 2 hours and, thereafter, was further stirred at room temperature for 18 hours. By adding 100 g of water to the resulting reaction mixture, a crystal was precipitated. Then, after the precipitated crystal was recovered by filtering operation, the obtained substance was washed with 10 g of water and 20 g of diethyl ether, and further dried to obtain 9.0 g of a white crystal. It was confirmed by ¹H-NMR that the resulting white crystal is N,N′-bis(2,6-dibromo-4-fluorophenyl)ethanediamide. Yield: 76%

¹H-NMR (δ/ppm, DMSO-d6, tetramethylsilane standard): 7.83 (d, J=8.1 Hz, 4H), 10.84 (s, 2H)

After a 200 mL autoclave made of stainless was charged with 3.0 g of the above-obtained N,N′-bis(2,6-dibromo-4-fluorophenyl)ethanediamide and 50 mL of BH3.tetrahydrofuran (1M solution), the mixture was heated and stirred at 75° C. for 16 hours. After cooled to room temperature, the reaction liquid was added in portions to a mixed liquid of 80 g of methanol and 5 g of 35% hydrochloric acid, and stirred. Lower-boiling substances were distilled off from the resulting reaction liquid, and 100 g of methanol was further added to the residue, and lower-boiling substances were distilled off again to obtain 2.7 g of a white crystal. It was confirmed by ¹H-NMR that the resulting crystal is N,N′-bis(2,6-dibromo-4-fluorophenyl)-1,2-ethanediamine hydrochloride. Yield: 80%

¹H-NMR (δ/ppm, DMSO-d6, tetramethylsilane standard): 3.30 (s, 4H), 5.99 (br, 2H), 7.61 (d, J=8.1 Hz, 4H)

A 50 mL flask replaced with nitrogen was charged with 1.1 g of the above-obtained N,N′-bis(2,6-dibromo-4-fluorophenyl)-1,2-ethanediamine hydrochloride and 20 g of triethyl orthoformate. After the resulting mixture was refluxed for 3 hours, the mixture was cooled to room temperature to precipitate a crystal. Then, after the precipitated crystal was recovered by filtering operation, the obtained substance was washed with 5 g of tetrahydrofuran and dried to obtain 560 mg of a white crystal. It was confirmed by ¹H-NMR that the resulting crystal is 1,3-bis(2,6-dibromo-4-fluorophenyl)imidazolinium chloride. Yield: 55%

¹H-NMR (δ/ppm, DMSO-d6, tetramethylsilane standard): 4.55 (s, 4H), 8.08 (d, J=8.1 Hz, 4H), 9.71 (s, 1H)

### Example 8

A 50 mL Schlenk tube replaced with nitrogen was charged with 3.0 g of 3-methylthiopropanal, 865 mg of paraformaldehyde, 166 mg of 1,3-bis(2,6-dibromophenyl)imidazolinium chloride obtained in Example 1 and 6 g of tetrahydrofuran. The resulting mixture was warmed to 60° C., and 40 mg of 1,8-diazabicyclo[5,4,0]-7-undecene was added under stirring, and then, the resulting mixture was stirred at 60° C. for 3 hours. The resulting reaction mixture was cooled to room temperature, thereby a reaction mixture containing 4-(methylthio)-2-oxo-1-butanol was obtained. As a result of analysis by a gas chromatography internal standard method, a yield of 4-(methylthio)-2-oxo-1-butanol was 75%, and 3-methylthiopropanal was recovered at 23%.

### Example 9

According to the same manner as that of Example 8 except that 114 mg of 1,3-bis(2,6-dichlorophenyl)imidazolinium chloride was used in place of 166 mg of 1,3-bis(2,6-dibromophenyl)imidazolinium chloride in Example 8, a reaction mixture was obtained. As a result of analysis by a gas chromatography internal standard method, a yield of 4-(methylthio)-2-oxo-1-butanol was 54%, and 3-methylthiopropanal was recovered at 34%.

### Example 10

According to the same manner as that of Example 8 except that 106 mg of 1,3-bis(2,4,6-tribromophenyl)imidazolinium chloride synthesized in Example 2 and 22 mg of 1,8-diazabicyclo[5,4,0]-7-undecene were used in place of 166 mg of 1,3-bis(2,6-dibromophenyl)imidazolinium chloride in Example 8, a reaction mixture was obtained. As a result of analysis by a gas chromatography internal standard method, a yield of 4-(methylthio)-2-oxo-1-butanol was 32%, and 3-methylthiopropanal was recovered at 67%.

### Example 11

A 50 mL Schlenk tube replaced with nitrogen was charged with 1.0 g of 3-methylthiopropanal, 290 mg of paraformaldehyde, 40 mg of 1,3-bis[(2,6-diisopropyl)-4-nitrophenyl]imidazolinium chloride obtained in Example 3 and 2 g of tetrahydrofuran. The resulting mixture was warmed to 40° C., and 15 mg of 1,8-diazabicyclo[5,4,0]-7-undecene was added under stirring, and then, the resulting mixture was stirred at 40° C. for 3 hours. The resulting reaction mixture was cooled to room temperature, thereby a reaction mixture containing 4-(methylthio)-2-oxo-1-butanol was obtained. As a result of analysis by a gas chromatography internal standard method, a yield of 4-(methylthio)-2-oxo-1-butanol was 12%, and 3-methylthiopropanal was recovered at 83%.

### Comparative Example 1

According to the same manner as that of Example 8 except that 123 mg of 1,3-bis(2,6-diisopropylphenyl)imidazolinium chloride (corresponding to a compound in which R³ and R⁴ are an aryl group having two electron withdrawing groups in an imidazolinium salt represented by the formula (1)) was used in place of 166 mg of 1,3-bis(2,6-dibromophenyl)imidazolinium chloride in Example 8, a reaction mixture was obtained. As a result of analysis by a gas chromatography internal standard method, a yield of 4-(methylthio)-2-oxo-1-butanol was 6%, and 3-methylthiopropanal was recovered at 55%.

### Example 12

A 50 mL Schlenk tube replaced with nitrogen was charged with 200 mg of 3-methylthiopropanal, 300 mg of 35% formalin, 10 mg of 1,3-bis(2,6-dibromophenyl)imidazolinium chloride obtained in Example 1 and 1 g of toluene. The resulting mixture was warmed to 60° C., and a mixed liquid of 3 mg of 1,8-diazabicyclo[5,4,0]-7-undecene and 100 mg of toluene was added under stirring, and then, the resulting mixture was stirred at 60° C. for 3 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 55%, and 3-methylthiopropanal was recovered at 20%.

### Example 13

A 50 mL Schlenk tube replaced with nitrogen was charged with 1.0 g of 3-methylthiopropanal, 1.2 g of 35% formalin, 70 mg of 1,3-bis(2,4,6-tribromophenyl)imidazolinium chloride obtained in Example 2 and 3 g of toluene. The resulting mixture was warmed to 60° C., and a mixed liquid of 15 mg of 1,8-diazabicyclo[5,4,0]-7-undecene and 100 mg of toluene was added under stirring, and then, the resulting mixture was stirred at 60° C. for 3 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 60%, and 3-methylthiopropanal was recovered at 35%.

### Example 14

A 50 mL Schlenk tube replaced with nitrogen was charged with 1.14 g of 3-methylthiopropanal, 1.9 g of 35% formalin, 40 mg of 1,3-bis(2,4,6-tribromophenyl)imidazolinium chloride obtained in Example 2 and 3 g of toluene. One gram of dry ice was added thereto, and an evolved carbon dioxide gas was removed to give a normal pressure. The resulting mixture was warmed to 60° C., and a mixed liquid of 9 mg of 1,8-diazabicyclo[5,4,0]-7-undecene and 100 mg of toluene was added under stirring, and then, the resulting mixture was stirred at 50° C. for 2 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 36%, and 3-methylthiopropanal was recovered at 63%.

### Example 15

A 50 mL Schlenk tube replaced with nitrogen was charged with 1.14 g of 3-methylthiopropanal, 620 mg of paraformaldehyde, 40 mg of 1,3-bis(2,4,6-tribromophenyl)imidazolinium chloride obtained in Example 2 and 3 g of toluene. One gram of dry ice was added thereto, and an evolved carbon dioxide gas was removed to get a normal pressure. The resulting mixture was warmed to 50° C., and a mixed liquid of 9 mg of 1,8-diazabicyclo[5,4,0]-7-undecene and 100 mg of toluene was added under stirring, and then, the resulting mixture was stirred at 50° C. for 2 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 56%, and 3-methylthiopropanal was recovered at 43%.

### Example 16

A 50 mL stainless reaction tube was cooled in a dry ice/methanol bath under a nitrogen atmosphere, 200 mg of 3-methylthiopropanal, 50 mg of 1,3-bis(2,6-dibromophenyl)imidazolinium chloride obtained in Example 1, 1.5 g of dry ice, and a mixed liquid of 15 mg of 1,8-diazabicyclo[5,4,0]-7-undecene and 500 mg of tetrahydrofuran were added thereto. After the reaction tube was sealed, the mixture was stirred at 40° C. for 3 hours. A pressure of the reaction tube was raised to 1.0 MPa. By cooling the resulting reaction mixture to room temperature, a reaction mixture containing 1,6-dimethylthio-4-hydroxy-3-hexanone was obtained. As a result of analysis by a gas chromatography area percentage method, a yield of 1,6-dimethylthio-4-hydroxy-3-hexanone was 85%, and 3-methylthiopropanal was recovered at 7%. Identification of 1,6-dimethylthio-4-hydroxy-3-hexanone was performed by GC-MS. MS (m/z): 208 (M+)

### Example 17

A 50 mL Schlenk tube replaced with nitrogen was charged with 1.0 g of propanal, 126 mg of 1,3-bis(2,4,6-tribromophenyl)imidazolinium chloride obtained in Example 2 and 1.5 g of tetrahydrofuran. The resulting mixture was warmed to 40° C., and a mixed liquid of 26 mg of 1,8-diazabicyclo[5,4,0]-7-undecene and 500 mg of tetrahydrofuran was added under stirring, and then, the resulting mixture was stirred at 40° C. for 2 hours. By cooling the resulting reaction mixture to room temperature, a reaction mixture containing 4-hydroxy-3-hexanone was obtained. As a result of analysis by a gas chromatography internal standard method, a yield of 4-hydroxy-3-hexanone was 11%, and propanal was recovered at 70%.

### Example 18

A 50 mL Schlenk tube replaced with nitrogen was charged with 1.2 g of 3-methylthiopropanal, 500 mg of paraformaldehyde, 50 mg of 1,3-bis[(4-dodecyl-2,6-dibromo)phenyl]imidazolinium chloride obtained in Example 4 and 3 g of toluene. Then, 0.5 g of dry ice was added thereto, and an evolved carbon dioxide gas was removed to get a normal pressure. The resulting mixture was warmed to 50° C., and a mixed liquid of 8 mg of 1,8-diazabicyclo[5,4,0]-7-undecene and 100 mg of toluene was added under stirring, and then, the resulting mixture was stirred at 50° C. for 2 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 75%, and 3-methylthiopropanal was recovered at 16%.

### Example 19

A 50 mL Schlenk tube replaced with nitrogen was charged with 1.14 g of 3-methylthiopropanal, 1.3 g of 35% formalin, 50 mg of 1,3-bis[(4-dodecyl-2,6-dibromo)phenyl]imidazolinium chloride obtained in Example 4 and 3 g of toluene. Then, 0.5 g of dry ice was added thereto, and an evolved carbon dioxide gas was removed to get a normal pressure. The resulting mixture was warmed to 50° C., and a mixed liquid of 9 mg of 1,8-diazabicyclo[5,4,0]-7-undecene and 100 mg of toluene was added under stirring, and then, the resulting mixture was stirred at 50° C. for 8 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 34%, and 3-methylthiopropanal was recovered at 61%.

### Example 20

A 50 mL Schlenk tube replaced with nitrogen was charged with 1.05 g of 3-methylthiopropanal, 1.2 g of 35% formalin, 30 mg of 1,3-bis(4-methyl-2,6-dibromophenyl)imidazolinium chloride obtained in Example 5 and 3 g of toluene. Then, 0.5 g of dry ice was added thereto, and an evolved carbon dioxide gas was removed to get a normal pressure. The resulting mixture was warmed to 50° C., and a mixed liquid of 8 mg of 1,8-diazabicyclo[5,4,0]-7-undecene and 100 mg of toluene was added under stirring, and then, the resulting mixture was stirred at 50° C. for 4 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 16%, and 3-methylthiopropanal was recovered at 78%.

### Example 21

A 50 mL Schlenk tube replaced with nitrogen was charged with 907 mg of 3-methylthiopropanal, 1.1 g of 35% formalin, 30 mg of 1,3-bis(4-t-butyl-2,6-dibromophenyl)imidazolinium chloride obtained in Example 6 and 3 g of toluene. Then, 0.5 g of dry ice was added thereto, and an evolved carbon dioxide gas was removed to get a normal pressure. The resulting mixture was warmed to 50° C., and a mixed liquid of 7 mg of 1,8-diazabicyclo[5,4,0]-7-undecene and 100 mg of toluene was added under stirring, and then, the resulting mixture was stirred at 50° C. for 2 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 34%, and 3-methylthiopropanal was recovered at 65%.

### Example 22

A 50 mL Schlenk tube replaced with nitrogen was charged with 1.00 g of 3-methylthiopropanal, 0.88 g of 35% formalin, 33 mg of 1,3-bis(2,6-dibromo-4-fluorophenyl)imidazolinium chloride obtained in Example 7 and 2 g of toluene. Then, 1 g of dry ice was added thereto, and an evolved carbon dioxide gas was removed to get a normal pressure. The resulting mixture was warmed to 60° C., and a mixed liquid of 7.5 mg of 1,8-diazabicyclo[5,4,0]-7-undecene and 100 mg of toluene was added under stirring, and then, the resulting mixture was stirred at 50° C. for 2 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 55%, and 3-methylthiopropanal was recovered at 30%.

### Example 23

A 100 mL reaction container replaced with nitrogen was charged with 10.0 g of 3-methylthiopropanal, 3.45 g of paraformaldehyde, 1.10 g of 1,3-bis(2,4,6-tribromophenyl)imidazolinium chloride obtained in Example 2 and 20 g of tetrahydrofuran. The resulting mixture was warmed to 45° C., and a carbon dioxide gas was introduced under stirring. A mixed liquid of 0.21 g of 1,8-diazabicyclo[5,4,0]-7-undecene and 5.25 g of toluene was added dropwise over 2.5 hours, and the resulting mixture was further stirred at 50° C. for 2 hours. By cooling the resulting reaction mixture to room temperature, a reaction mixture containing 4-(methylthio)-2-oxo-1-butanol was obtained. As a result of analysis by a gas chromatography internal standard method, a yield of 4-(methylthio)-2-oxo-1-butanol was 88%, and 3-methylthiopropanal was recovered at 5%. The resulting reaction mixture was distilled under reduced pressure to obtain 7.1 g of 4-(methylthio)-2-oxo-1-butanol (boiling point from 85 to 94° C./46.7 Pa, content 94%).

### Example 24

A 50 mL Schlenk tube replaced with nitrogen was charged with 500 mg of benzaldehyde, 290 mg of paraformaldehyde, 35 mg of 1,3-bis(2,4,6-tribromophenyl)imidazolinium chloride obtained in Example 2 and 3 g of toluene. Then, 0.5 g of dry ice was added thereto, and an evolved carbon dioxide gas was removed to get a normal pressure. The resulting mixture was warmed to 50° C., and a mixed liquid of 7 mg of 1,8-diazabicyclo[5,4,0]-7-undecene and 100 mg of toluene was added under stirring, and then, the resulting mixture was stirred at 50° C. for 2 hours. By cooling the resulting reaction mixture to room temperature, a reaction mixture containing 2-hydroxy-1-phenylethanone was obtained. As a result of analysis by a gas chromatography internal standard method, a yield of 2-hydroxy-1-phenylethanone was 15%, and benzaldehyde was recovered at 84%.

### INDUSTRIAL APPLICABILITY

According to the present invention, an α-hydroxyketone compound can be produced easily and effectively.

1. A process for producing an α-hydroxyketone compound, comprising a stirring step of stifling one or more aldehyde compounds or polymers thereof in the presence of a base and an imidazolinium salt represented by the formula (1): 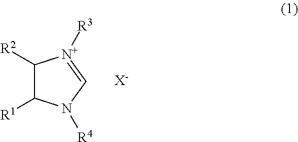
wherein
R¹ and R² each independently represent a hydrogen atom, an alkyl group optionally having a substituent or an aryl group optionally having a substituent, or R¹ and R² are bound to each other to form a ring together with carbon atoms to which they bind,
R³ and R⁴ each independently represent an aryl group having one or more electron withdrawing groups, and
X⁻ represents an anion.

2. The process according to claim 1, wherein the base is at least one base selected from the group consisting of organic bases, alkali metal salts and alkaline earth metal salts.

3. The process according to claim 1, wherein R³ and R⁴ each are independently an aryl group having at least one group selected from the group consisting of a halogen atom, a nitro group a cyano group, an alkoxycarbonyl group, an acyl group, and a sulfo group.

4. The process according to claim 1, wherein the imidazolinium salt represented by the formula (1) is an imidazolinium salt represented by the formula (1A): 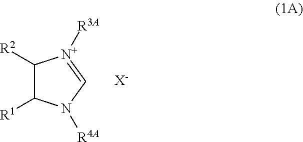
wherein
R¹, R² and X⁻ mean the same as defined above, and
R^{3A} and R^{4A} each independently represent a 2,6-dichlorophenyl group optionally having a substituent, a 2,6-dibromophenyl group optionally having a substituent, a 2,6-bis(trifluoromethyl)phenyl group optionally having a substituent, a 2,6-diphenylphenyl group or a 2,6-diisopropyl-4-nitrophenyl group.

5. The process according to claim 1, wherein the stirring step is performed in the presence of carbon dioxide.

6. The process according to claim 1, wherein the aldehyde compound or polymer thereof is an aldehyde compound represented by the formula (2): 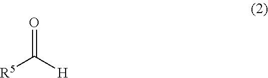
wherein
R⁵ represents a hydrogen atom or an alkyl group optionally having a substituent
or a polymer thereof.

7. The process according to claim 1, wherein the aldehyde compounds or polymers thereof are an aldehyde compound represented by the formula (2): 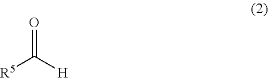
wherein
R⁵ represents a hydrogen atom or an alkyl group optionally having a substituent
or a polymer thereof and an aldehyde compound represented by the formula (4): 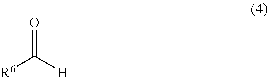
wherein
R⁶ is different from R⁵, and represents a hydrogen atom, an alkyl group optionally having a substituent, an aryl group optionally having a substituent or a heteroaryl optionally having a substituent
or a polymer thereof.

8. The process according to claim 7, wherein R⁵ in the formula (2) is an alkyl group optionally having a substituent.

9. The process according to claim 7, wherein the aldehyde compound represented by the formula (2) or a polymer thereof is 3-methylthiopropanal.

10. The process according to claim 8, wherein the aldehyde compound represented by the formula (4) or a polymer thereof is formaldehyde or a polymer thereof, and wherein the stirring step is performed in the presence of water.

11. An imidazolinium salt represented by the formula (1A): 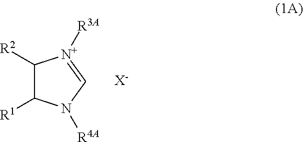
wherein
R¹, R² and X⁻ mean the same as defined above,
R^{3A} and R^{4A} each independently represent a 2,6-dichlorophenyl group optionally having a substituent, a 2,6-dibromophenyl group optionally having a substituent, a 2,6-bis(trifluoromethyl)phenyl group optionally having a substituent, a 2,6-diphenylphenyl group or a 2,6-diisopropyl-4-nitrophenyl group.

12. The imidazolinium salt according to claim 11, wherein R¹ and R² are both a hydrogen atom, and R^{3A} and R^{4A} each independently represent a 2,6-dibromophenyl group, a 4-tert-butyl-2,6-dibromophenyl group, a 4-dodecyl-2,6-dibromophenyl group, a 2,4,6-tribromophenyl group, a 4-fluoro-2,6-dibromophenyl group or a 2,6-diisopropyl-4-nitrophenyl group.

13. The imidazolinium salt according to claim 11, wherein R¹ and R² are both a hydrogen atom, and R^{3A} and R^{4A} each independently represent a 2,6-dibromophenyl group, a 2,4,6-tribromophenyl group, a 4-fluoro-2,6-dibromophenyl group or a 2,6-diisopropyl-4-nitrophenyl group.
